(19) United States
(12) Patent Application Publication
(10) Pub. No.: US 2023/0260426 A1
(43) Pub. Date: Aug. 17, 2023

(54) A Training Device, System and Method

(71) Applicant: Sanofi, Paris (FR)

(72) Inventors: Michael Helmer, Frankfurt am Main (DE); Martin Vitt, Frankfurt am Main (DE)

(21) Appl. No.: 18/011,230

(22) PCT Filed: Jun. 23, 2021
(86) PCT No.: PCT/EP2021/067149

§ 371 Date: Dec. 19, 2022

(30) Foreign Application Priority Data
Jun. 25, 2020 (EP) 20315320.0

(51) Int. Cl. G09B 23/28; G09B 5/02; A61M 5/32; A61M 5/315; A61M 5/20

(52) U.S. Cl. CPC G09B 23/285; A61M 5/20; A61M 5/32; A61M 5/31548; G09B 5/02

(57) ABSTRACT

A system including a training drug delivery device and a user device is provided. The training drug delivery device includes a body, a cap, a delivery activation button, a controller, a memory, a wireless unit for communicating with the user device, a drive for simulating a haptic response of a drug delivery device, and at least one sensor for measuring attachment of the cap and depression of the delivery activation button. The user device includes a controller, a memory, and a wireless unit. The user device is configured to connect to the training drug delivery device, receive sensor measurements from the training drug delivery device, and provide, based at least partly on the received sensor measurements, the user with feedback on handling the training drug delivery device.

### CROSS REFERENCE TO RELATED APPLICATIONS

The present application is the national stage entry of International Patent Application No. PCT/EP2021/067149, filed on Jun. 23, 2021, and claims priority to Application No. EP 20315320.0, filed on Jun. 25, 2020, the disclosures of which are incorporated herein by reference.

### TECHNICAL FIELD

The present disclosure relates to a training device, training system, and a method of operating the training device and system.

### BACKGROUND

Drug delivery devices, such as pen-type drug delivery devices, insulin pumps, blood glucose monitoring devices, auto-injectors and the like, have application where regular injection by persons without formal medical training occurs. This is increasingly common among patients having diabetes where self-treatment enables such patients to conduct effective management of their diabetes.

For instance, a pre-filled disposable insulin pen can be used as a drug delivery device. Alternatively, a re-usable pen may be used. A re-usable pen allows replacement of an empty medicament cartridge by a new one. Either pen may come with a set of needles that are replaced before each use. The insulin dose to be injected can then for instance be manually selected at the insulin pen by turning a dosage dial and observing the actual dose from a dosage window of the insulin pen. The dose is then injected by inserting the needle into a suitable skin portion and pressing an injection button of the insulin pen.

Typically, a user of the drug delivery device is a patient who does not have any formal medical training. It is desirable to provide the user with training in use of the drug delivery device. Different users prefer different ways of learning and being instructed, and respond to different training modes. Available training tools (e.g. existing written manuals or videos), however, may not provide training which a particular user would find sufficiently comprehensive and/or easy to remember.

### SUMMARY

In a first aspect, a system comprising a training drug delivery device and a user device is provided, the training drug delivery device comprising a body, a cap and a delivery activation button, the training drug delivery device further comprising a controller, a memory, a wireless unit for communicating with the user device, a drive for simulating a haptic response of a drug delivery device, and at least one sensor for measuring attachment of the cap and depression of the delivery activation button; the user device comprising a controller, a memory and a wireless unit; the training drug delivery device being adapted to: connect, using the wireless unit, to the user device; and transmit sensor measurements to the user device; the user device being configured to: connect, using the wireless unit, to the training drug delivery device; receive sensor measurements from the training drug delivery device; and provide, based at least partly on the received sensor measurements, the user with feedback on handling the training drug delivery device.

In various embodiments, one or more of the following features may be used:
the training drug delivery device comprises at least one of the following: an injection mechanism, a dosage dial for selecting a dose, a dosage window for showing the selected dose and a needle;
the training drug delivery device further comprising at least one sensor for measuring one or more of the following: position of the training drug delivery device, orientation of the training drug delivery device, torque exerted on the dosage dial, force exerted on the injection mechanism, attachment of the cap covering at least a part of the body, attachment of the cap covering the needle and attachment of the needle;
the feedback is provided to the user using augmented reality or virtual reality provided by the user device;
the training drug delivery device comprises at least one window-shaped region defined on the body, the window-shaped region being adapted to support an augmented reality / virtual reality projection;
the user device is configured to project information onto a drug window or a dosage window or both;
the user device is adapted to transmit parameters of haptic response to the training drug delivery device, and the training drug delivery device is adapted to receive the parameters of haptic response from the user device;
the haptic response parameters comprise parameters defining response of the dosage dial and/or the injection mechanism;
the haptic response parameters define clicking or resistance of the dosage dial in response to being turned and/or the injection mechanism in response to being pressed, and wherein the resistance is provided by the drive;
the drug delivery device is configured to: detect, using the at least one sensor, a user’s handling of the training drug delivery device; transmit, using the wireless module; the sensor indications to the user device, the sensor indications corresponding to the user’s handling of the training drug delivery device, the sensor indications comprising at least one of position of the training drug delivery device, torque exerted on the dosage dial, force exerted on the injection mechanism, attachment of the cap covering at least a part of the body, attachment of the cap covering the needle, attachment of the needle, and wherein the user device is configured to: indicate, in response to receiving the sensor indications, to the user whether the in user’s handling of the training drug delivery device is correct;
the training drug delivery device comprises a container adapted to be filled with liquid, or wherein the training drug delivery device is adapted to receive a drug cartridge;
the system further comprises a training pad simulating a user’s skin;
the haptic response of a drug delivery device is based on parameters of the haptic response received from the user device
the training drug delivery device further comprises a needle shield and a needle shield activation button.

In a second aspect, a training drug delivery device is provided, the training drug delivery device comprising a body, a cap and a delivery activation button, the training drug delivery device further comprising a controller, a memory, a wireless unit for communicating with the user device, a drive for simulating a haptic response of a drug delivery device, and at least one sensor for measuring attachment of the cap and depression of the delivery activation button; the training drug delivery device being adapted to: connect, using the wireless unit, to a user device; and transmit sensor indications to the user device.

In a third aspect, a training drug delivery device is provided, the training drug delivery device being adapted for use with any of the embodiments of the system described in connection with the first aspect.

In a fourth aspect, a user device is provided, the user device comprising a controller, a memory and a wireless unit; the user device being configured to: connect, using the wireless unit, to the training drug delivery device; receive sensor indications from the training drug delivery device; and transmit parameters of haptic response to the training drug delivery device; and provide, based at least partly on the received sensor measurements, the user with feedback on using the training drug delivery device.

In a fifth aspect, a user device is provided, the user device being adapted for use with any of the embodiments of the system described in connection with the first aspect

With the features described above, the system, device and method can bring the following advantages.

The user training may be standardized, with the same training provided to each user (patient). The training may not be influenced by the particular health care professional providing the training or other individual circumstances. The user may be able to go back easily to parts of the training which they feel they need to repeat (e.g. because the user did not understand the particular part of the training properly). This may encourage users to complete the training properly, with full understanding of handling of the drug delivery device.

Once the user completes a training, a certificate may be issued in an automated way. The certificate may serve as a proof for the user and/or for the drug manufacturer that the user has been properly trained in use of the drug delivery device.

The above-described system allows enhancements in differentiation tests. Usually, the differentiation tests (i.e. tests whether the drug delivery device is distinct from other similar drug delivery devices and thus recognizable for the users) are part of a design process designing the drug delivery device, and may be only concluded with a small to medium-sized sets of users. With the system, device and method described above, each user may be subject to differentiation test. The results may be collected for statistical purposes. The differentiation tests may help the user to learn the differences between the user’s drug delivery device and other drug delivery devices (either belonging to the same user, or belonging to other users), thus lowering the risk of injecting a wrong drug and/or a wrong dose. With the help of augmented reality or virtual reality, the differentiation tests may be carried out with each user under adverse conditions (e.g. simulation of the appearance of the drug delivery device under dimmed light). In this way, the risk of the user making an error in selecting the correct drug delivery device under adverse conditions may be lowered.

### BRIEF DESCRIPTION OF THE FIGURES

FIG. 1a is an exploded view of a training drug delivery device;

FIG. 1b shows a system of a training drug delivery device, a user device and augmented reality (AR) or virtual reality (VR) glasses;

FIG. 2 is a schematic view of the electronic components of the training drug delivery device;

FIG. 3 is a flow chart of an example training using the system of the training drug delivery device, the user device and/or the augmented reality (AR) or virtual reality (VR) glasses;

FIG. 4 is a flow chart of drug delivery device recognition training from FIG. 3;

FIG. 5 is a flow chart of drug delivery device handling training from FIG. 3;

FIG. 6 is a flow chart of drug and drug device training from FIG. 3;

FIG. 7 is a flow chart of supervised first use of drug delivery device from FIG. 3;

FIG. 8 is a flow chart of refilling the drug delivery device training from FIG. 3;

FIG. 9 is a flow chart of an example set up and training using the system of the training drug delivery device, the user device and/or the augmented reality (AR) or virtual reality (VR) glasses;

FIGS. 10 to 14 show examples of distinguishing features.

### DETAILED DESCRIPTION

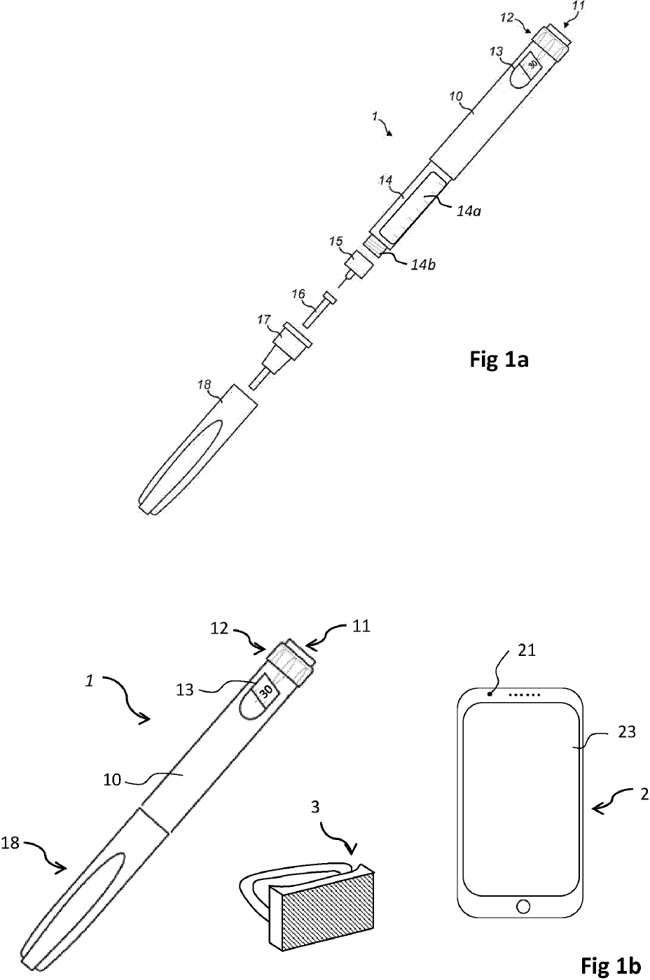

FIG. 1a is an exploded view of a training drug delivery device 1, which may for instance represent Sanofi’s Solostar (TM) insulin injection pen.

FIG. 1b is a schematic view of a training system comprising the training drug delivery device 1 and two user devices 2, 3: a mobile device 2 and augmented reality (AR) or virtual reality (VR) glasses 3.

The user device 2, 3 may be for example an electronic device, such as mobile phone or tablet (more generally, a mobile device). The user device 2 may be a desktop PC, a laptop, or a tablet. The user device 2 comprises a screen 23. At least one camera 21 is associated with the user device 2. The camera 23 may be integral part of the user device 2. The camera 21 may be a front camera or a back camera. The camera 21 may be an external camera, such as a webcam.

The user device 2, 3 may be for example AR/VR glasses 3. The AR/VR glasses 3 may be a head mount for a mobile device 2. The AR/VR glasses 3 may be a device independent of a mobile device 2. If the AR/VR glasses 12 are independent of a mobile device, the mobile device 2 may be optional.

The training drug delivery device 1 is shown in detail in FIG. 1a. The training drug delivery device 1 comprises a housing 10. In general, the housing 10 is provided with a delivery activation button. The delivery activation button is often provided in the form an injection mechanism (e.g. an injection button) 11. The body 10 of the training drug delivery device 1 of the figures further comprises a dosage dial 12, a dosage window 13, and a container region 14.

A needle 15 can be affixed to the container region 14. The needle 15 may be a real disposable needle, or it may be a training needle, which, for example, has the same overall shape but lacks the sharp end, and is incapable of penetrating the skin (not shown).

The housing 10 is further provided with a cap 18 which covers the container region 14 when the training drug delivery device 1 is not in use. When the training drug delivery device 1 is not in use, the needle 15 is not attached. Before the needle 15 is affixed to the housing 1, the cap 18 is removed.

The housing 10 is provided with a container region 14 to which the needle 15 can be affixed. In one embodiment, the container region 14 contains a container. The container may be refillable, and may be adapted to be filled with a suitable liquid, e.g. water or training substitute of insulin. In one embodiment, the container is not refillable. In an embodiment, the container region 14 may be adapted to receive a training medicament cartridge (not shown), for example a training insulin cartridge. In one embodiment, the container region 14 does not contain a container.

The container region 14 may comprise a drug window 14a. The drug window 14a may be a real window (i.e. an opening through which a user can see the container and/or the contents of the container). In an embodiment, the drug window is a simulated window. A simulated window may be a window-shaped region defined on the container region 14. The window-shaped region 14a may be filled with a neutral color (e.g. grey, green or blue) to support keying or other suitable video, augmented reality or virtual reality projection. The window-shaped region 14a may thus be adapted to interact with an augmented reality or virtual reality set (described below).

In an embodiment, the dimensions and weight of the training drug delivery device 1 are the same as a real drug delivery device. In an embodiment, the dimensions, colors and design of each of the elements of the training drug delivery device 1 correspond to the dimensions, colors and design of the elements of a real drug delivery device 1. In an embodiment, the training drug delivery device 1 is provided in neutral colors (e.g. grey, green or blue) to support keying or other suitable video, augmented reality or virtual reality projection and the user device 2, 3 is adapted to project colors and design of the elements of a real drug delivery device onto the training drug delivery device 1.

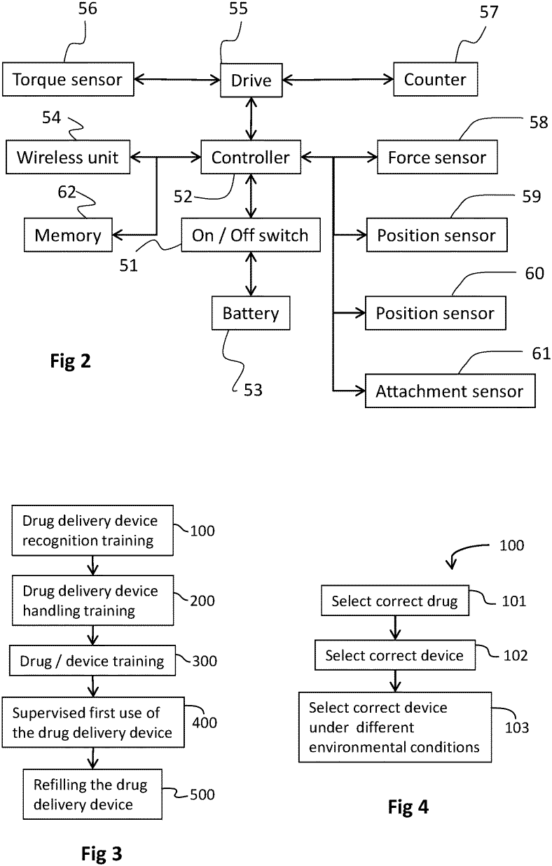

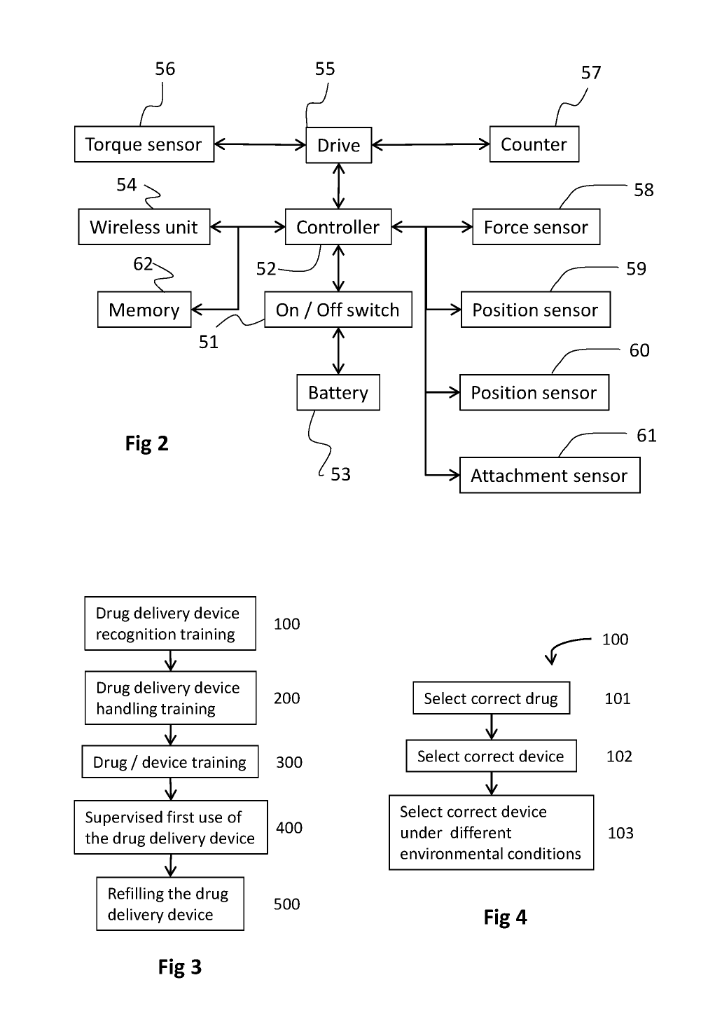

An appropriate dose of a medicament can be selected by turning the dosage dial 12. In the training drug delivery device 1, the dosage dial 12 may be connected to a drive 55 (see FIG. 2), which provides a haptic response which simulates the response provided by a real drug delivery device (e.g. an insulin pen). More generally, the drive 55 may provide a mechanical force and/or resistive force simulation. The drive 55 may act as a mechanical force and/or mechanical resistance generator.

The dosage dial 12 may be also connected to a torque sensor 56 and a counter 57 and is adapted to detect the dialed or redialed dose. In response, the user device 2, 3 may indicate to the user whether the selected dose is correct.

The selected dose may be displayed via dosage window 13, for instance an insulin dose in multiples of so-called International Units (IU), wherein one IU is the biological equivalent of about 45.5 micrograms of pure crystalline insulin (1/22 mg). An example of a selected dose displayed in dosage window 13 may, for instance, be 30 IUs, as shown in FIGS. 1. The dosage window 13 may be a real window with a digital or analogue display. In an embodiment, the dosage window 13 is a simulated window. A simulated window may be a window-shaped region defined close to the dosage dial 12. The window-shaped region may be filled with a neutral color (e.g. grey, green or blue) to support keying or other suitable video, augmented reality or virtual reality projection. The window-shaped region may thus be adapted to interact with the AR / VR glasses 3.

Turning the dosage dial 12 may cause a mechanical clicker to provide haptic and acoustic response to the user. In an embodiment, the haptic and acoustic response provided to the user is the same as would be provided by a real drug delivery device. In an embodiment, turning the dosage dial 12 causes the distance between the dosage dial 12 and the body 10 of the drug delivery device 1 to be increased.

The housing 10 comprises an injection mechanism 11. The injection mechanism may be e.g. an injection button 11. In one embodiment, pressing the injection button 11 provides the user with haptic and acoustic response to the user. For example, the distance between the dosage dial 12 and the body 10 of the drug delivery device 1 is decreased upon pressing of the injection button 11, as would be the case with a real drug delivery device. Alternatively or in addition, the mechanical clicker may also produce a sound upon pressing of the injection button 11, similarly to a real drug delivery device. In an embodiment, the haptic and acoustic response provided to the user is the same as would be provided by a real drug delivery device. Pressing the injection button 11 simulates delivery of a drug.

In a preferred embodiment, the training drug delivery device 1 does not contain a real drug. The training drug delivery device 1 may contain water or placebo. The training drug delivery device 1 may contain any suitable liquid which is not pharmaceutically active (e.g. saline). The training drug delivery device 1 may not contain any liquid; for example, the training drug delivery device 1 may contain air, or there may be no container provided, and the training drug delivery device 1 may contain no fluid.

The needle 15 is protected by an inner needle cap 16 and an outer needle cap 17. The needle 15 can be screwed or pressed onto a needle end 14b of the container region 14. The needle end 14b may be provided with an attachment sensor 61, which measures the position and the correct attachment of the needle 15.

FIG. 2 is a schematic representation of electronic features of the training drug delivery device 1. The training drug delivery device 1 has an on/off switch 51, a controller 52, a memory 62 and a battery 53. The training drug delivery device 1 has a wireless unit 54, for example a Bluetooth unit or a Wi-Fi unit. The wireless unit 54 is capable of connecting the training drug delivery device 1 to the user device 2, 3.

The training drug delivery device 1 preferably has a drive 55, a torque sensor 56 and a counter 57. The drive 55, the torque sensor 56 and the counter 57 are connected to the dosage dial 12 and the injection button 11. The drive 55 may be e.g. a motor, a generator, a stepper producing a DC pulse for each unit dialed on the dosage dial 12, or an electromechanical (E/M) drive.

When the dosage dial 12 is turned to select a dose and/or when the injection button 11 is pressed to release the dose, the drive 55 may provide haptic response to the user. For example, clicking and/or resistance similar to or same as a real drug delivery device, when a real dosage dial is turned and/or a real injection button pressed.

Preferably, the training drug delivery device 1 is capable of providing the user with similar or same haptic response (feedback) as a real drug delivery device. As this depends on which particular type of a real drug delivery device the user needs to be trained for, the training drug delivery device 1 may be programmed to simulate several different drug delivery devices. The user device 2, 3 may transmit a set of haptic response parameters to the training drug delivery device 1, based e.g. on a selection of the device the user is trained for. Preferably, the haptic response parameters comprise parameters defining response of the dosage dial 12, the injection button 11 and other features. Preferably, these parameters are adjustable in accordance with the drug delivery device the user is being trained for and/or the drug the user is being trained for and/or other considerations.

For example, the force necessary to press the injection button 11 may vary with a particular device and/or with viscosity of a particular drug; based on which device the user is trained for and/or which drug the user is being trained for, the force necessary to press the injection button 11 in the training drug delivery device may be adjusted accordingly.

For example, the force necessary to turn the dosage dial 12 and thus select a dose may depend on the particular drug delivery device. Based on the particular drug delivery device the user is being trained for, the force may be adjusted accordingly.

In one example, the injection button 11 may be blocked, i.e. set to be impossible to press, to simulate a blocked needle. In such case, the user may be required to replace the needle.

The drive 55 may be adapted to lower and lock a needle shield. In some drug delivery devices, the needle 15 is a safety needle (not shown). A safety needle is a needle which, after use, is protected by a shield. The shield may be for example spring loaded, and may be lowered and locked in place once the injection of the dose of the drug has been finished. The drive 55 may be adapted to release the needle shield. The release of the needle shield may happen upon a command from the user device 2, 3.

The drive 55 may be adapted to further simulate one or more of the following:
the force required to press injection button 11 as it depends on viscosity of a particular drug and a particular drug delivery device;
the time required to inject a full dose of the drug by the drug delivery device as it depends on viscosity of particular drug and a particular drug delivery device;
the primary pack stopper movement for pen and auto injectors (i.e. a maximum dose back stop corresponding to the maximum extent to which the pen can be dialed (e.g. 80 units) and a last dose back stop, which is a mechanical stop which prevents a user from dialing in more dose than is left in the cartridge);
clicks which in some drug delivery devices are provided to the user as a feedback, to indicate that the drug started to be delivered (first click) and that the delivery of the drug is finished (second click);
clicks and force required to turn the dosage dial 12;
locking and/or unlocking a needle shield lock provided in some drug delivery devices, which falls and locks into place to protect the (used) needle 15 following a successful injection of a full dose of the drug.

The drive 55 may be further adapted to reset the training drug delivery device 1 into its original position after manipulation by the user (training of the user) is finished.

The torque sensor 56 and the counter 57 may be adapted to measure the dose selected by the user by turning the dosage dial 12. The information obtained from the torque sensor 56 and the counter 57 may be transmitted to the user device 2, 3 for further processing. For example, the user device 2, 3 may be adapted to assess, based on a pre-stored or pre-programmed data, whether the user selected the correct dose, in a correct manner.

The training drug delivery device 1 further comprises one or more sensors 58-61. For example, the training drug delivery device 1 may have a force sensor 58, a position sensor 59, a position sensor 60, and an attachment sensor 61.

The force sensor 58 may be connected to the injection button 11, detecting the force with which the user presses down the injection button 11. The information obtained from the force sensor 58 may be transmitted to the user device 2, 3 for further processing. For example, the user device 2, 3 may be adapted to assess whether the user presses the injection button 11 with correct force and for correct duration of time to release the full dose of a medicament. For example, the time required to release the full dose of a medicament may be between 5 seconds and 15 seconds. The time may depend on a particular drug delivery device and/or a particular drug. Preferably, the time necessary for releasing the full dose of a medicament is stored in the user device 2, 3 for the particular combination of drug and drug delivery device.

A number of position sensors 59, 60 may be provided. In FIG. 2, two position sensors 59, 60 are shown. There may be one position sensor, or they may be more position sensors than two. The position sensors 59, 60 are adapted to detect the position of the training drug delivery device 1. The position sensors 59, 60 measure the position of the training drug delivery device 1 and changes in this position (indicating the user handling of the training drug delivery device 1). The position of the training drug delivery device 1 detected by the position sensors 59, 60 is transmitted to the user device 2, 3. The user device 2, 3 may use the information transmitted from the position sensors 59, 60 in AR/VR processing. For example, the position of the training drug delivery device 1 may be used in correct aligning of the information displayed over the dosage window 13, the drug window 14a, etc.

At least one of the position sensors 59, 60 may be adapted to detect removal and/or reattachment of the cap 18. At least one of the position sensors 59, 60 may be adapted to detect attachment and/or removal and/or lowering of the shield of the needle 15. A suitable sensor adapted to detect removal and/or reattachment of the cap 18 may be e.g. a switch or a capacitive sensor. A suitable sensor adapted to detect attachment and/or removal and/or lowering of the shield of the needle 15 may be e.g. a switch, a capacitive sensor, a proximity sensor or a contact based sensor (e.g. a piezoelectric sensor).

The attachment sensor 61 may be adapted to detect the position of the needle 15 on the needle end 14b of the container region 14. The detected position of the needle 15 on the needle end 14b of the container region 14 may be transmitted to the user device 2, 3, where it is used to assess whether the user has attached the needle 15 in a correct place and/or in a correct manner. A suitable sensor adapted to detect the position of the needle 15 on the needle end 14b of the container region 14 may be e.g. a switch or a capacitive sensor.

The user device 2, 3 may provide various training or support options. The training provided may for example focus on recognition of the correct drug delivery device to be used (e.g. a correct choice between several different drug delivery devices); handling of the drug delivery device; training related to the drug to be injected with the drug delivery device; and assisted (supervised) first use of the drug delivery device.

In an embodiment, the training may be organized in successive blocks. An example is illustrated in FIG. 3. The user is first offered a drug delivery device recognition training 100. After successfully completing the drug delivery device recognition training 100, the user proceeds to a drug delivery device handling training 200. After successfully completing the drug delivery device handling training 200, the user proceeds to drug / device training 300. After successfully completing the drug / device training 300, the user proceeds to supervised first use of the drug delivery device 400.

In some embodiments, the drug / device training 300 may be provided before the drug delivery device recognition training 100. In some embodiments, the drug / device training 300 may be provided before the drug delivery device training 200. In some embodiments, the drug / device training 300 may be provided before step 202 and step 203 of the drug delivery device training 200. In some embodiments, the drug / device training 300 may be provided multiple times.

Any one of the blocks 100, 200, 300, 400, 500 may be provided together with (before or after) any other of the blocks 100, 200, 300, 400, 500. Any one of the blocks 100, 200, 300, 400, 500 may be optional.

FIG. 4 illustrates an example of drug delivery device recognition training 100. The drug delivery device recognition training 100 may use the training drug delivery device 1 described above. The drug delivery device recognition training 100 may use the user device 2, 3. In use, the user may be first prompted to select a correct drug 101 (based on his condition, his immediate situation and previous advice and/or instructions by a health care professional). For example, a user who is a diabetic patient is asked to select between several types of insulin (meal-time insulin, long-acting insulin, etc.).

Once the step of selecting a correct drug 101 is successfully completed, the user proceeds to selection of a correct device training 102. In the selection of a correct device training 102, the user may be prompted to identify the correct drug delivery device. The user device 2, 3 may offer a choice of several (e.g. three) different devices to choose from. Based on characteristics of the drug delivery device such as labels, dimensions, shapes, colors, etc., the user selects the drug delivery device. The selection may be made using the screen of the mobile device 2.

To provide the selection of a correct device training 102, the user device 2, 3 may cooperate with the training drug delivery device 1. The training drug delivery device 1 is placed on a surface, e.g. a table. The position of the training drug delivery device 1 is detected by the user device 2, 3, using the position sensors 59, 60 of the training drug delivery device 1. In the augmented reality or virtual reality displayed by the user device 2, 3, the correct device may be projected over the training drug delivery device 1. The training drug delivery device 1 may be displayed among a number (e.g. two) of different drug delivery devices. The user is then requested to identify the correct drug delivery device; the user may do so by reaching for the training drug delivery device 1.

If the user’s selection is incorrect, the drug delivery device recognition training 100 may be suspended. The user device 2, 3 may then provide the user with additional training or guidance. For example, the user device 2, 3 may show the user some or all features that distinguish the correct drug delivery device from the drug delivery device chosen by the user. Such features may be for example shapes of specific components of the respective drug delivery devices, colors of specific components of the respective drug delivery devices, dimensions of the respective drug delivery devices, etc. For example, the drug delivery devices may differ in label, the color of the body, the color of the dosage dial, the color of the button, and the shape and position of the cap and features provided on the cap. All these differences may be pointed out to the user in response to the user selecting the wrong drug delivery device.

After the user successfully completes the selection of a correct device training 102, the user may be either asked or offered to complete a selection of a correct device under different environmental conditions training 103. The selection of a correct device under different environmental conditions training 103 may present the user with a similar training to the selection of a correct device training 102. The drug delivery devices presented to the user are, however, presented in different sets of simulated conditions. For example, the drug delivery devices may be presented as if they were in a dimmed light environment, with some or all of the distinguishing features (e.g. labels, dimensions, shapes, colors etc.) being less distinct and thus less easy to notice and evaluate for the user.

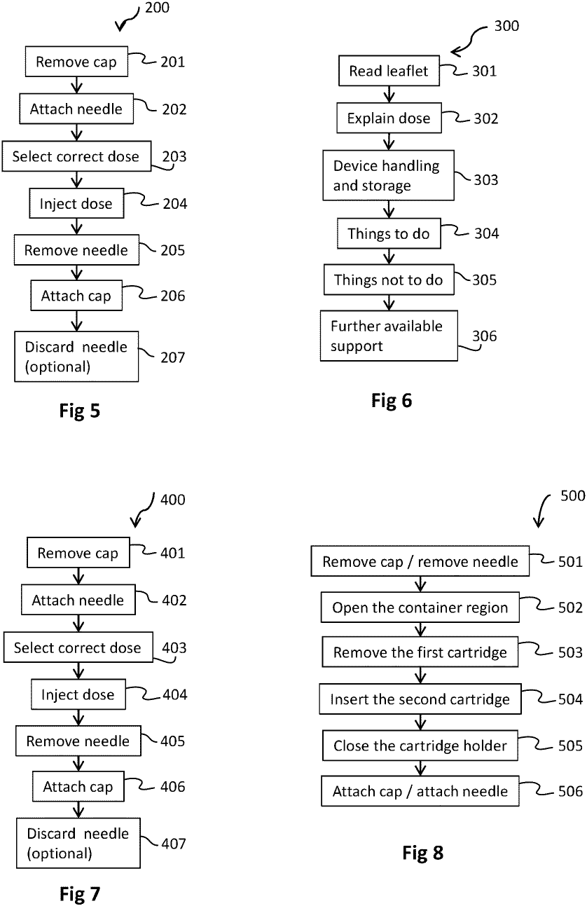

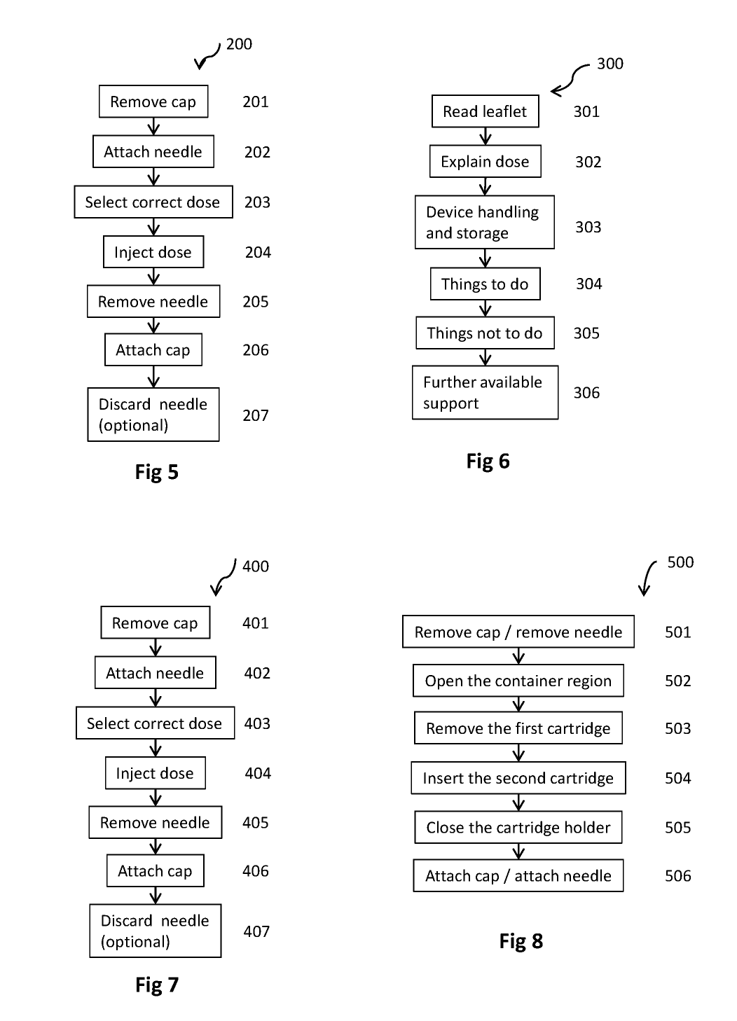

FIG. 5 shows an example of a drug delivery device handling training 200. The user may be offered or required to complete the drug delivery device handling training 200 after successfully completing the drug delivery device recognition training 100. The user may be offered or required to complete the drug delivery device handling training 200 as a standalone training (i.e. without further conditions such as completing any other training first).

In the drug delivery device handling training 200, the user device 2, 3 may prompt the user to complete, step-by-step, a typical procedure necessary to inject the drug and store the drug delivery device safely. The user device 2, 3 cooperates with the training drug delivery device 1 to accomplish the drug delivery device handling training 200.

In each of the steps described below, the user may be guided through the respective step by the user device 2, 3. For example, the user may be shown an instruction video on how to accomplish each task. Once the video has been played back to the user, the user may be prompted to try and accomplish the task themselves.

An example drug delivery device handling training 200 starts with the user having the training drug delivery device 1 in their hand and in view of the camera 21 of the user device 2, 3. Optionally, the user device 2, 3 projects information onto the training drug delivery device 1. For example, if the dosage window 13 and/or the drug window 14a are simulated windows, the user device 2, 3 may project a selected dose and/or a simulated content of the drug contained in the container region 14.

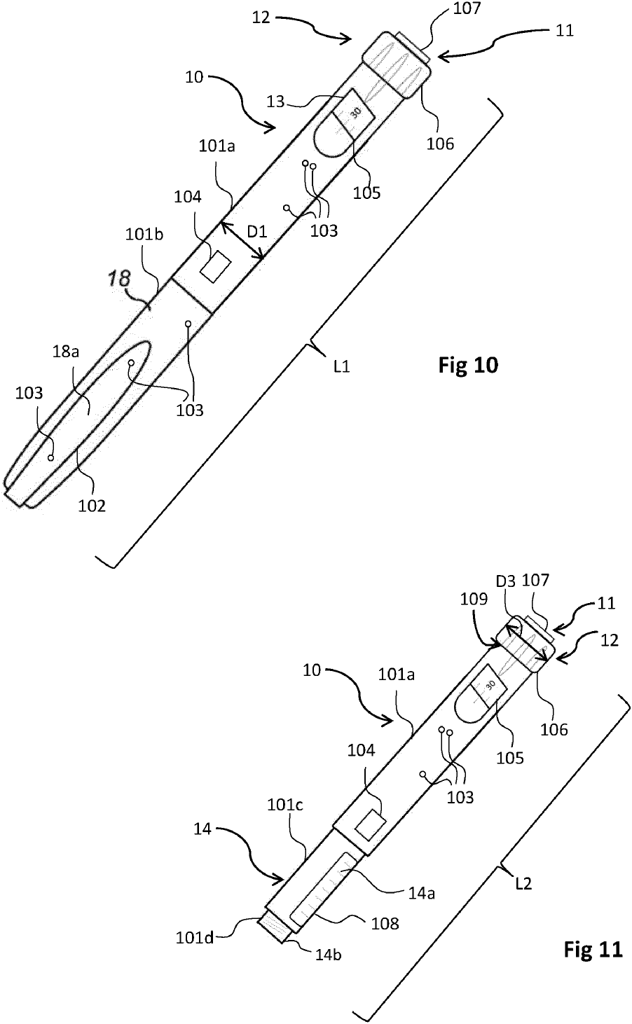

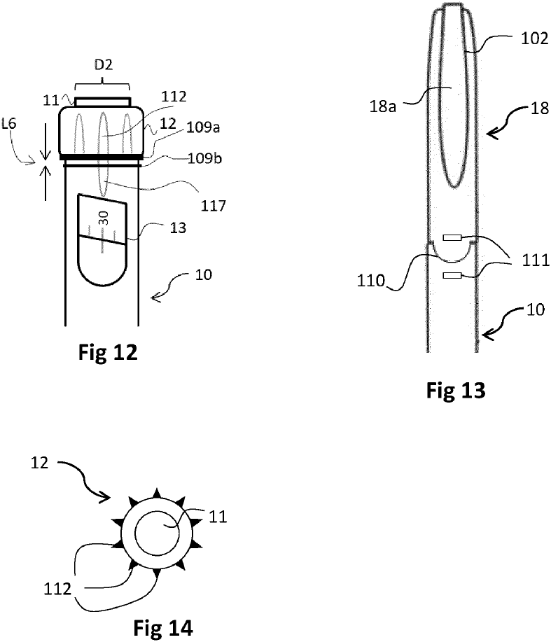

The user device 2, 3 prompts the user to remove the cap 201 of the training drug delivery device 1. When the user accomplishes this task, the position sensors 59, 60 may indicate to the user device 2, 3 that the cap 18 has been removed from the training drug delivery device 1. Alternatively or in addition, the user device 2, 3 may use an image of the training drug delivery device 1 captured by the camera 21. The position of the body 10 and/or the cap 18 in the captured image may be determined based on one or more distinctive features provided on the cap 18 and/or the body 10 of the training drug delivery device 1. The distinctive features are described below in connection to FIGS. 10-14.

In response to detecting the position of the cap 18 by the user device 2, 3, the user device 2, 3 may indicate to the user that the task has been successfully accomplished (for example by displaying green color, a tick mark icon, or in any other suitable way).

The user device 2, 3 may then prompt the user to attach the needle 202. Attaching the needle 202 may require several sub-steps, for example attaching the needle 15 protected by an inner needle cap 16 and an outer needle cap 17 and subsequent removing of the outer needle cap 17 and the inner needle cap 16. The attachment sensor 61 may indicate to the user device 2, 3 whether the task has been successfully accomplished. The attachment sensor 61 may be also able to indicate most common errors, e.g. the needle 15 being only partially attached. Alternatively or in addition, the user device 2, 3 may use gesture control to assess whether the needle 15 has been successfully attached. Alternatively or in addition, the user device 2, 3 may use an image captured by the camera 21 to assess whether the needle has been successfully attached, similarly to what has been described above in connection with step 201. The user device 2, 3 may then indicate the status (e.g. successful completion of the task) to the user, as described above.

Once the needle 15 is in place, the user may be prompted by the user device 2, 3 to select a correct dose 203. The user may be guided towards turning the dosage dial 12 until a correct dose is displayed in the dosage window 13. The turning of the dosage dial 12 and thus the selected dose is detected by the torque sensor 56 and the counter 57 and transmitted to the user device 2, 3, which may display this information as augmented reality / virtual reality in the dosage window 13.

When turning the dosage dial 12 and selecting the correct dose, the drive 55 preferably provides haptic response (haptic feedback) expected in this situation from a real drug delivery device (e.g. clicking and/or resistance same as a real drug delivery device when the dosage dial of the real drug delivery device is turned).

The user device 2, 3 may determine that the user has finished turning the dosage dial 12 and selecting the correct dose once the user does not turn the dosage dial 12 for a predetermined period of time (e.g. 3 seconds). Alternatively or in addition, the user may be asked to confirm to the user device 2, 3 that they have finished turning the dosage dial 12 and selecting the correct dose.

Once the user has finished turning the dosage dial 12 and selecting the correct dose, the user device 2, 3 assesses the selected dose and indicates whether the selected dose is correct. The assessment and indication may be accomplished as described above. In particular, the user device 2, 3 may use any one or more of sensor data, captured images and gesture control to assess whether the task has been completed successfully, and the user device 2, 3 may then indicate a correct completion of the task using any means described above.

Once the user accomplishes selecting a correct dose, the user may be prompted to inject the dose 204.

In one embodiment, the user presses the injection button 11 of the training drug delivery device 1, without sticking the needle 15 into the portion of the patient’s skin. The training drug delivery device 1 preferably provides haptic response (haptic feedback) expected in this situation from a real drug delivery device (e.g. resistance dependent on the viscosity of the drug, same as in a real drug delivery device when the dosage dial 12 is set to a given dose and the given drug is selected).

In an embodiment the training drug delivery device 1 may contain a liquid, to simulate a drug contained in the container region 14.

In one embodiment, a training pad (not shown) is provided. The training pad is a device which simulates a portion of the skin of a user. It may be attachable to the user’s body in an area in which the user is most likely to inject the drug. For example, the training pad may be adapted to be attached to the user’s thigh or around the user’s waist. Using a training pad enhances the haptic response provided to the user, and it may provide more realistic training to the user. The training pad may be made of absorbent material.

When the needle 15 is stuck into the training pad and then the injection button 11 is pushed, in one embodiment a liquid (which corresponds to a simulated insulin dose displayed in dosage window 13) is ejected from the training drug delivery device 1 into the training pad. In one embodiment, air is ejected from the training drug delivery device 1 into the training pad the needle 15 is stuck into the training pad and then the injection button 11 is pushed. In another embodiment, the needle 15 is stuck into a training pad and the injection button 11 is pressed without any movement of mechanical parts, such as plunger, and without any dispensing of fluid or liquid.

Pressing the ejection button 11 of the training drug delivery device 1 may cause a mechanical click sound, which is different from the sounds produced when using dosage dial 12.

During the injection of dose 204, the training drug delivery device 1 together with the user device 2, 3 preferably monitors at least one of the following: the force exerted by the user to press the injection button 11; the time the user spends pressing the injection button 11; the angle under which the user sticks the needle 15 in the training pad. Based on the data thus measured, the user device 2, 3 assesses whether the user injected the dose correctly. The data is preferably measured as described above, i.e. the user device 2, 3 may use any one or more of sensor data, captured images and gesture control to assess whether the task has been completed successfully. The successful completion of dose injection 204 may be indicated by the user device 2, 3, as described above.

Once the dose injection step 204 is successfully completed, the user may be required or prompted to proceed to remove the needle 205. Removing the needle 205 may require several sub-steps. For example, the user may be required to attach the inner needle cap 16 and the outer needle cap 17 before detaching the needle 15. The attachment sensor 61 indicates to the user device 2, 3 whether the task has been successfully accomplished. Alternatively or in addition, the user device 2, 3 may use any one or more of sensor data, captured images and gesture control to assess whether the task has been completed successfully. The user device 2, 3 may then indicate the status (e.g. successful completion of the task) to the user, as described above.

Once the needle 15 has been successfully removed, the user may be required or prompted to attach the cap 206. The position sensors 59, 60 may indicate to the user device 2, 3 that the cap 18 has been attached to the training drug delivery device 1. Alternatively or in addition, the user device 2, 3 may use any one or more of sensor data, captured images and gesture control to assess whether the task has been completed successfully. The user device 2, 3 may then indicate the status (e.g. successful completion of the task) to the user, as described above.

The user device 2, 3 may be adapted to detect if the user is trying to attach the cap 18 while the needle 15 is still attached, and alert the user.

In some embodiments, the user may be required to also safely dispose of the used needle 207. A container for disposing of sharps (not shown) may be provided. The user may be required to discard the needle 15 in the container and confirm that this had been done. Alternatively or in addition, the user device 2, 3 may use captured images and gesture control to assess whether the task has been completed successfully. The user device 2, 3 may then indicate the status (e.g. successful completion of the task) to the user, as described above.

The drug delivery device training 200 described above may have several levels of difficulty. For example, a user who handles a drug delivery device for the first time ever may be reminded of the next step in each phase of the training, may be provided with more guidance when handling the training drug delivery device 1, etc. For example, a user who has reviewed the training several times, or is more experienced in handling drug delivery devices, may be provided with very little guidance, or no guidance at all. Similar considerations apply to the processes described in FIGS. 6-8.

The training drug delivery device 1 together with the user device 2, 3 is preferably adapted to detect mistakes and errors. For example, the user may try to select the dose and subsequently inject the drug without the needle 15 mounted, or the user may try to attach the cap 18 with the needle 15 still attached. In such situation, the user device 2, 3 may interrupt the training (with or without saving the position, as appropriate), and provide the user with more guidance (e.g. playing an instruction video to them again). Similar considerations apply to the processes described in FIGS. 6-8.

Once the user successfully completes the training, the user device 2, 3 may issue a certificate of successful completion of the training. Successful completion of the training may be assessed based on a threshold number of trainings accomplished without a mistake (e.g. if the user goes through the whole drug delivery device training 200 three times without a mistake), completing the drug delivery device training 200 with minimal or no guidance at least once, etc.). Similar considerations apply to the processes described in FIGS. 6-8.

An exemplary drug training 300 is shown in FIG. 6. After each step, the user may be asked whether they understood the training content and/or confirm that they would like to proceed to the next step.

In the first step, the user may be provided with an assisted reading of the drug leaflet 301. Assistance provided to the user may include loud reading. Assistance provided to the user may be supervised reading. For example, the assistance may comprise assessing whether the user has read and/or understood the content of the leaflet. Assessment of whether the user has read and/or understood the content of the leaflet may be based e.g. on monitoring the user’s eye movements. The user’s eye movements may mean one or more of the following: the direction of the user’s gaze, the speed of the user’s eye movements, the direction of the user’s eyes movements, the time spent by the user focusing on (reading or watching) a content, the timing of the user’s blinking, the rate of the user’s blinking, duration of pupil fixation, number of pupil fixations, view path, pupil diameter and stress load; and dwell time (per content).

In the next step 302, a dosage regime and/or a dose may be explained to the user. The explanation may be based on the leaflet information, on the advice of a health care professional, or both.

In the next step 303, the user may receive explanation about correct handling of the device. For example, the user may be shown an instruction video explaining all steps 201-207 and 401-407. The user may receive instruction on how to remove the cap of the device, attach the needle, select correct dose, inject the dose (incl. in which area of the user’s body to inject the dose and how to handle the skin before, during and after the injection), remove the needle, attach the cap, and discard the needle.

The user may further receive explanation about correct storing of the drug delivery device. For example, the drug delivery device may be required to be stored in a specific temperature range (e.g. between 2° C. and 8° C.), under specific light condition (e.g. in a dark place, away from direct sunlight), etc.

In the next steps 304, 305, the user may receive further explanations about how to handle and/or how not to handle the drug delivery device. For example, the user may be warned against the most common mistakes in handling the device. For example, the device may be required to be stored between 2° C. and 8° C. and should be warmed up at a room temperature for at least 45 min before use; the user may be advised about this requirement, and warned against trying to warm the drug delivery device up e.g. using direct sunlight or putting the drug delivery onto a heater.

In step 306, the user may receive an indication of a further support available. For example, the user may receive a list of health care centers dealing specifically with their type of medical condition, pharmacies that sell the drug delivery device and/or the drug the user uses, or persons which may be able to help with troubleshooting.

FIG. 7 shows a typical procedure for an assisted first use of a drug delivery device 400. The steps 401-407 correspond to the steps 201-207. The main difference between the process 200 and the process 400 is that unlike process 200, process 400 is carried out with a real drug delivery device, loaded with a real drug which the user injects into their skin rather than in a training pad.

The assistance provided by the user device 2, 3 during supervised first use of the drug delivery device 400 may be similar to the assistance described above in connection with FIG. 5. For example, the user device 2, 3 may prompt user to perform each step 401-407. The user may be given an opportunity to review training content (e.g. instruction video) associated with a/each step before proceeding with performing the step.

The user device 2, 3 may use specific distinctive features to locate the position and orientation of the drug delivery device, and may use this data to supervise whether the user performs the steps 401-407 correctly, whether the user handles the drug delivery device in a correct manner, etc. Alternatively or in addition, the user may be asked to indicate when they complete each step 401-407 so that the user device 2, 3 may proceed to the next step. To perform this function, the user device 2, 3 may monitor the user’s eye movements (described above in connection with step 301). Alternatively or in addition, the user device 2, 3 may monitor the distinctive features of the training drug delivery device 1 (described below).

In particular, in step 401, the user may be asked to remove the cap of the drug delivery device 401. Based on recognition of distinctive features provided on the body of the device and the cap, the user device 2, 3 may detect that the step has been accomplished. Alternatively or in addition, the user may indicate that the cap has been removed and the user is ready for the next step.

In step 402, the user may be prompted to attach the needle. If the user is not sure how to proceed, the user may receive additional instruction (e.g. an instruction video may be played back to them). If the step of attaching the needle requires several sub-steps (e.g. attaching the needle and subsequently removing an outer needle cap and an inner needle cap), the user device 2, 3 may guide the user through all the sub-steps. The user device 2, 3 may remind user to perform the sub-steps in the correct order (e.g. first attach the needle, then remove the needle caps rather than removing the needle caps and then attaching the needle). As in step 401, the user device 2, 3 may detect that the step has been accomplished and/or the user may indicate that the needle has been attached.

In step 403, the user may be prompted to select the correct dose. If the user is not sure how to proceed, the user may receive additional instruction (e.g. an instruction video may be played back to them). The user device 2, 3 may be able to detect the selected dose based on recognition of the number displayed in the dosage window provided on the drug delivery device. Alternatively or in addition, the user may be asked to input this number in the user device 2, 3 for verification. Based on the selected dose, the user device 2, 3 may indicate whether the dose is correct or incorrect. In case the dose is incorrect, the user may be guided by the user device 2, 3 to select a correct dose.

In step 404, the user may be prompted to inject the selected dose. The user may be guided to avoid the most common mistakes. The user may be e.g. prompted to press the injection button all the way down. The user may be guided to keep pressing the injection button for a certain amount of time (e.g. 5-15 seconds) and/or keep the needle in their skin for a certain amount of time. The user may receive an indication from the user device 2, 3 once they can withdraw the needle from their skin and/or stop pressing the injection button. As in the previous steps, the user device 2, 3 may detect that the step has been accomplished and/or the user may indicate that the dose has been injected. For example, the user device 2, 3 may detect that after disappearing from view for a period of time, the drug delivery device returned into view.

In step 405, the user may be prompted to remove the needle. If the step of removing the needle requires several sub-steps (e.g. attaching an outer needle cap and an inner needle cap and subsequently removing the needle), the user device 2, 3 may guide the user through all the sub-steps. The user device 2, 3 may remind user to perform the sub-steps in the correct order (e.g. first attach the needle caps and then remove the needle rather than removing the needle and then attaching the needle caps). As in the previous steps, the user device 2, 3 may detect that the step has been accomplished and/or the user may indicate that the needle has been removed.

In step 406, the user may be asked to attach the cap of the drug delivery device. Based on recognition of distinctive features provided on the body of the device and the cap, the user device 2, 3 may detect that the step has been accomplished. Alternatively or in addition, the user may indicate that the cap has been attached.

In step 407, the user may be prompted to safely dispose of the used needle. The user may be e.g. guided to discard the used needle into a container for disposing of sharps.

FIG. 8 shows an example of training in refilling the drug delivery device 500. The example is described from the point of view of a drug delivery device 1 having a replaceable cartridge (not shown) positioned in the container region 14.

In step 501, the user may be prompted to either remove the cap 18 of the drug delivery device 1 (if the cap 18 is attached) or to remove the needle 15 (if the needle is attached). The user device 2, 3 may be adapted to recognize whether the cap 18 or the needle 15 is attached, and prompt the user accordingly. The user device 2, 3 may detect that the step has been accomplished and/or the user may indicate that the cap or the needle has been removed.

In step 502, the user device 2, 3 may prompt the user to open the container region 14. The two components of the container region 14 may be e.g. screwed, snap-fitted or press-fitted together. The user device 2, 3 may be adapted to guide the user and provide an indication how the container region 14 opens and closes, so that the user may accomplish the task. The user device 2, 3 may detect that the step has been accomplished and/or the user may indicate that the container region has been opened.

In step 503, the user may be prompted by the user device 2, 3 to remove a first cartridge (not shown) from the container region 14. The first cartridge may be considered empty or almost empty, or it may be considered containing a medicament which is no longer good to use (e.g. is cloudy). As in the previous steps, the user device 2, 3 may detect that the step has been accomplished and/or the user may indicate that the cartridge has been removed.

In step 504, the user may be prompted by the user device 2, 3 to insert a second cartridge (not shown). The user device 2, 3 may provide the user with support to help them to insert the cartridge in the correct way. To this end, the user device 2, 3 may indicate to the user distinguishing features provided on the cartridge, such as colored ends, one end being wider than the other end, etc. The user device 2, 3 may detect that the step has been accomplished and the cartridge has been correctly inserted. Alternatively or in addition, the user may indicate that the cartridge has been inserted, and optionally how the cartridge has been inserted.

In step 505, the user device 2, 3 may prompt the user to close the container region 14. The container region may be impossible to close if the cartridge is inserted incorrectly. The user device 2, 3 may detect such situation and advice the user that the cartridge needs to be placed correctly and/or may guide the user in how to remove and replace the cartridge correctly. The user device 2, 3 may detect that the step has been accomplished and/or the user may indicate that the container region has been closed.

In step 506, the user device 2, 3 may prompt the user to replace the cap 18 or to attach the needle 15 (and optionally proceed with any other training 100, 200, 300, 400), as appropriate.

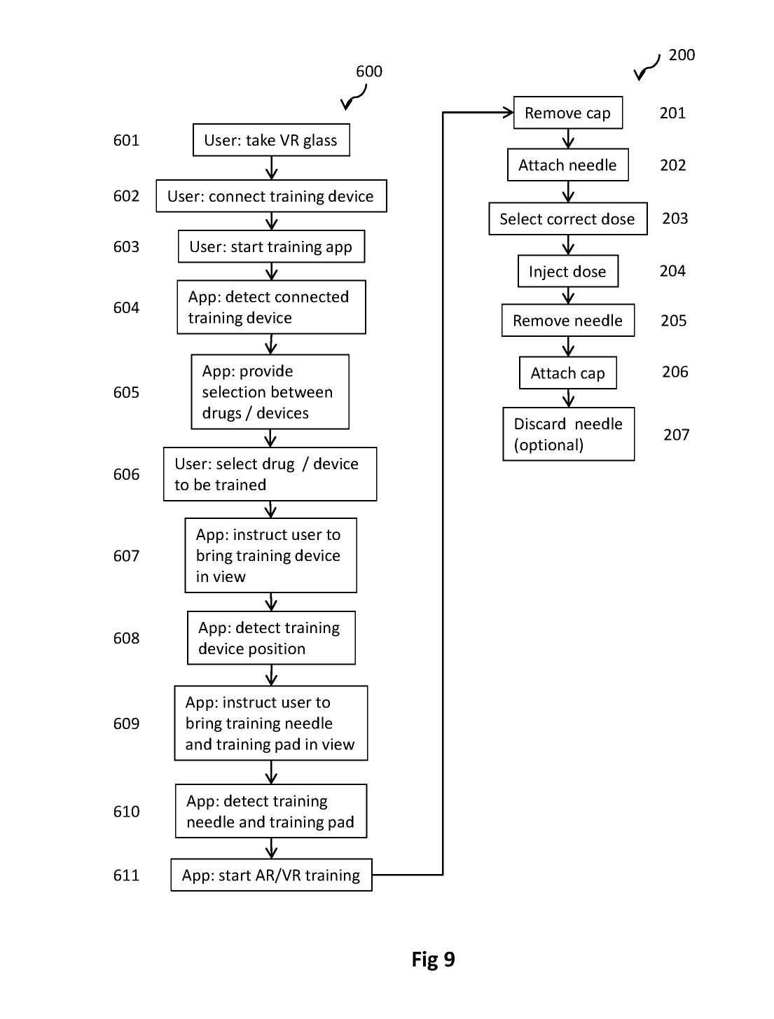

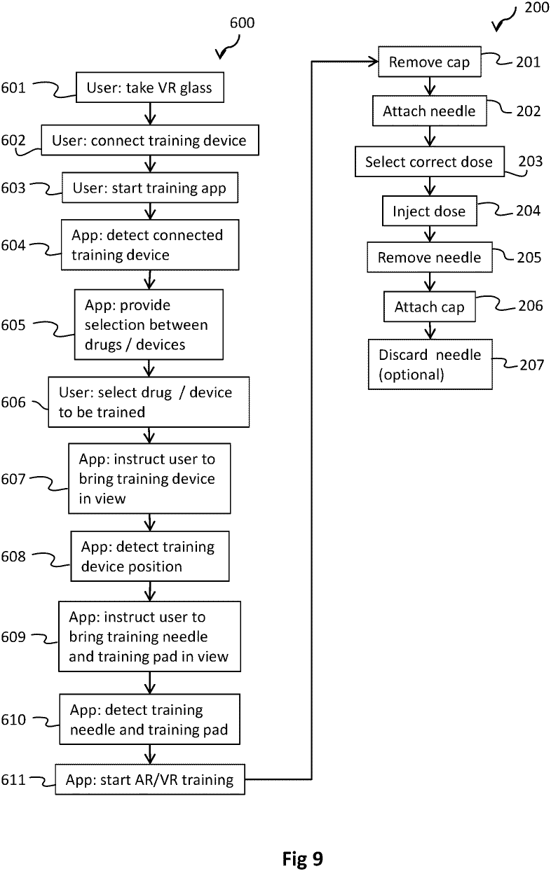

FIG. 9 shows an example of handling the system of the training drug delivery device 1 and the user device 2, 3. The process shown in FIG. 9 is divided into two main blocks, a set-up phase 600 and training 200. The training 200 is provided as an example only. The training 200 may be replaced with one or more of the drug delivery device recognition training 100; drug training 300; supervised first use 400; or refilling the drug delivery device 500.

In step 601, the user takes and activates the user device 2, 3.

In step 602, the user connects the training drug delivery device 1, using e.g. the wireless unit 54 of the training drug delivery device 1 and a corresponding wireless unit provided in the user device 2, 3 (not shown). The user device 2, 3 may be adapted to automatically detect the training drug delivery device 1. The user device 2, 3 may be adapted to automatically connect the training drug delivery device 1. The user device 2, 3 may connect the training drug delivery device 1 after receiving an approval from the user.

In step 603, the user starts a training application. The training application is a software application providing the user with the training and assistance in handling the training drug delivery device 1 and a real drug delivery device.

In step 604, the user device 2, 3, using the training app, detects the training drug delivery device 1.

In step 605, the user device 2, 3 may prompt the user to select a drug and/or a drug delivery device for the training. In subsequent step 606, the user indicates their selection.

For example, the user device 2, 3 may offer the user a selection of three different drugs: long-acting insulin; rapid-acting insulin; and GLP-1/Glu dual agonist. Based on the user’s selection, the user device 2, 3 provides training 200.

For example, the user device 2, 3 may offer the user a selection of three different drug delivery devices: Device A, delivering long-acting insulin; Device B, delivering rapid-acting insulin; and Device C, delivering GLP-1/Glu dual agonist. Based on the user’s selection, the user device 2, 3 provides training 200.

For example, based on the user’s selection, the user device 2, 3 may select the haptic response parameters of the training drug delivery device 1 (e.g. the response of the dosage dial 12 or the injection button 11 of the training drug delivery device 1 as these depend on device and drug, as described above) corresponding to the selected device and/or selected drug. The selected haptic response may be communicated from the user device 2, 3 to the training drug delivery device 1 using the wireless unit 54.

For example, based on the user’s selection, the user device 2, 3 may select appropriate AR/VR images and overlay these with the training drug delivery device 1, to simulate the contents of the dosage window 13, the drug window 14a, the colors and design of the selected drug delivery device, etc.

In step 607, the user device 2, 3 may instruct the user to bring the training drug delivery device 1 in front of the camera 21 and thus into view of the user device 2, 3. In step 608, the user device 2, 3 may detect the position, the orientation or both the position and the orientation of the training drug delivery device 1. The user device 2, 3 may detect the position and/or the orientation of the training drug delivery device 1. The detection may be based on data received from one or more sensors 58-61 of the training drug delivery device 1. Alternatively or in addition, the detection may be based on one or more distinctive features (described above).

In step 609, the user device 2, 3 may prompt the user to bring the needle 15 and the training pad (not shown) in front of the camera 21 of the user device 2, 3. In step 610, the user device 2, 3 may detect the needle 15 and the training pad. The detection may be based on any one or more of the following: distinct shapes of the needle 15 and the training pad; distinct colors of the needle 15 and/or the training pad; detecting a NFC tag provided in the needle 15 and/or the training pad; an attachment sensor provided in the needle 15.

The drug delivery device 1 has one or more distinctive features 101-112, L1-L6, D1-D3. The user device 2, 3 stores suitable software enabling the user device 2, 3 to identify the distinctive features 101-112, L1-L6, D1-D3 and compare them to a pre-determined set of distinctive features associated with a particular device. The user device 2, 3 therefore enables recognition of the drug delivery device 1. The distinctive features 101-112, L1-L6, D1-D3 may enable the user device 2, 3 to identify the status of the drug delivery device 1.

Preferably, the user device 2, 3 stores more than one distinctive feature 101-112, L1-L6, D1-D3 of a given drug delivery device 1. If the user device 2, 3 stores more than one distinctive feature 101-112, L1-L6, D1-D3 of a given drug delivery device 1, the drug delivery device 1 may be identified by the user device 2, 3 with more accuracy. If the user device 2, 3 stores more than one distinctive feature 101-112, L1-L6, D1-D3 of a given drug delivery device 1, it may be possible to recognize one or more of the following: position of the drug delivery device 1, orientation of the drug delivery device 1 with respect to the user device 2, 3, distance of the drug delivery device 1 from the user device 2, 3, etc. Alternatively or in addition, if the user uses the user device 2, 3 for capturing a video rather than a static picture, it may be possible to identify the movements of the drug delivery device 1, changes in its position, orientation, distance from the user device 2, 3 etc.

Example features which may serve as distinctive features 101-112, L1-L6, D1-D3 are shown in FIGS. 10-14. The distinctive features 101-112, L1-L6, D1-D3 may be for example:
outer shape 101a, 101b of the body 10 of the drug delivery device 1 and the cap 18 which is in place over the container region 14;
outer shape 101a, 101c, 101d of the body 10 of the drug delivery device 1 and the container region 14 (i.e. with the cap 18 removed);
outer shape of the body 10 of the drug delivery device 1, the container region 14 and the needle 15;
shape 102 of a latch 18a (the latch 18a being provided on the cap 18);
color of the of the body 10 of the drug delivery device 1;
dots or pixels 103 having a color different from the rest of the body 10 of the drug delivery device 1;
a code 104 such as data matrix code or QR code provided on the body 10, on the cap 18, or both on the body 10 and the cap 18;
the shape and/or color 105 of the dosage window 13;
the shape and/or color 106 of the dosage dial 12;
the shape and/or color 107 of the injection button 11;
the shape and/or color 108 of the drug window 14a;
the distance L6 between the dosage dial 12 and the body 10 of the drug delivery device 1;
closure features 110 on the cap 18 and the corresponding portion of the body 10 (see FIG. 13);
notches or protrusions 111 provided on the cap 18, the body 10, or both the cap 18 and the body 10 (see FIG. 13);
number and position of notches 112 on the dosage dial 12 (see FIG. 14);
the length L1 of the drug delivery device 1 including the cap 18 (see FIG. 10);
the length L2 of the drug delivery device 1 without the cap 18 and without the needle 15 (see. FIG. 11);
the length L3 of the drug delivery device 1 without the cap 18 and without the inner and outer needle caps 16-17 but with the needle 15 attached (not shown);
the length L4, L5 of the drug delivery device 1 without the cap 18 but with one or both of the inner and outer needle caps 16 -attached (not shown);
the diameter D1 of the body 10 of the drug delivery device 1 (see FIG. 10);
the diameter D2 of the injection button 11;
the diameter D3 of the dosage dial 12 (see FIG. 11);
the ratio of any two of the lengths L1-L6 and/or diameters D1-D3 (in general, any of the ratios Lx/Ly, Dx/Dy, or Lx/Dy, where x, y stand for the number of the respective length or diameter);
distances (not shown) between any two of the distinctive features 101-112 listed above and/or ratios of any two of these distances;
one or more NFC tags, e.g. a RFID tag, provided in the cap 18, the body 10 or both the cap 18 and the body 10 of the drug delivery device 1 (not shown).

The distinctive features 101-112, L1-L6, D1-D3 may be used alone or in combination. The user device 2, 3 may recognize one or more of the features in a given drug delivery device 1. Any one of the features 101-112, L1-L6, D1-D3 listed above can be used for drug delivery device recognition in combination with any other of the features 101-112, L1-L6, D1-D3. The user device 2, 3 may store any subset of the above listed distinctive features 101-112, L1-L6, D1-D3, including all the distinctive features 101-112, L1-L6, D1-D3. Recognition by the user device 2, 3 of any of the distinctive features 101-112, L1-L6, D1-D3 listed above can be combined with recognition by the user device 2, 3 of any other of the distinctive features 101-112, L1-L6, D1-D3. The drug delivery device 1 may be provided with any subset of the above listed distinctive features 101-112, L1-L6, D1-D3, including all the distinctive features 101-112, L1-L6, D1-D3. A drug delivery device 1 may be provided with any one of the features 101-112, L1-L6, D1-D3 listed above in combination with any other of the features 101-112, L1-L6, D1-D3.

The user device 2, 3 stores predefined distinctive features of a given drug delivery device 1 (such as any one of the distinctive features 101-112, L1-L6, D1-D3, a subset of the distinctive features 101-112, L1-L6, D1-D3 listed above, or all of the distinctive features 101-112, L1-L6, D1-D3 listed above). The user device 2, 3 is capable of comparing the distinctive features measured from the image or video captured by the camera 21 to the predefined distinctive features stored in the memory 25 of the user device 2, 3 to identify which drug delivery device 1 is presented to the camera 21.

In step 611, the user device 2, 3 starts the training. 200. The training 200 may be started upon verification of the user device 2, 3 that the training drug delivery device 1 is connected, set up and fully functional.

The process then continues to training 200. Training 200 and the steps 201-207 are discussed above. The training 200 may be replaced with one or more of the drug delivery device recognition training 100; drug training 300; supervised first use 400; or refilling the drug delivery device 500.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about -4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N- tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(w-carboxyheptadecanoyl)-des(B30) human insulin and B29-N-(w-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide / HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1-15. (canceled)

16. A system comprising:
a user device comprising a controller, a memory, and a wireless unit; and
a training drug delivery device comprising a body, a cap, a delivery activation button, a controller, a memory, a wireless unit for communicating with the user device, a haptic drive for simulating a haptic response of a real drug delivery device, and at least one sensor configured to measure (i) an attachment of the cap to the body and (ii) a depression of the delivery activation button relative to the body;
the training drug delivery device being configured to connect, using the wireless unit of the training drug delivery device, to the user device and transmit sensor measurements of the at least one sensor to the user device;
the user device being configured to (i) connect, using the wireless unit of the user device, to the training drug delivery device, (ii) receive the sensor measurements from the training drug delivery device, and (iii) provide, based at least partly on the received sensor measurements, a user with feedback on handling the training drug delivery device.

17. The system of claim 16, wherein the training drug delivery device comprises at least one of the following: an injection mechanism, a dosage dial for selecting a dose, a dosage window for showing the selected dose, or a needle.

18. The system of claim 17, wherein the at least one sensor is configured to measure one or more of the following: (i) a position of the training drug delivery device within an environment, (ii) an orientation of the training drug delivery device within the environment, (iii) a torque exerted on the dosage dial relative to the body, (iv) a force exerted on the injection mechanism relative to the body, (v) an attachment of the cap covering at least a part of the body, (vi) an attachment of the cap covering the needle, or (vii) an attachment of the needle to the training drug delivery device.

19. The system of claim 16, wherein the feedback is provided to the user using augmented reality or virtual reality provided by the user device.

20. The system of claim 19, wherein the training drug delivery device comprises at least one window-shaped region defined on the body, the window-shaped region being configured to support an augmented reality or virtual reality projection.

21. The system of claim 20, wherein the user device is configured to project information onto at least one of a drug window of the training drug delivery device or a dosage window of the training drug delivery device.

22. The system of claim 16, wherein:
the user device is configured to transmit haptic response parameters of the haptic response to the training drug delivery device, and
the training drug delivery device is configured to receive the haptic response parameters of the haptic response from the user device.

23. The system of claim 22, wherein the haptic response parameters comprise parameters defining a response of at least one of a dosage dial of the training drug delivery device or an injection mechanism of the training drug delivery device.

24. The system of claim 23, wherein the haptic response parameters define a clicking or resistance of the dosage dial in response to being turned relative to the body of the training drug delivery device or a clicking or resistance of the injection mechanism in response to being pressed relative to the body of the training drug delivery device, the clicking or resistance of the dosage dial or the injection mechanism being provided by the haptic drive of the training drug delivery device.

25. The system of claim 16, wherein the training drug delivery device is configured to:
detect, using the at least one sensor, a user’s handling of the training drug delivery device based on one or more sensor indications comprising at least one of (i) a position of the training drug delivery device within an environment, (ii) a torque exerted on a dosage dial of the training drug delivery device relative to the body of the training drug delivery device, (iii) a force exerted on an injection mechanism of the training drug delivery device relative to the body of the training drug delivery device, (iv) an attachment of the cap covering at least a part of the body of the training drug delivery device, (v) an attachment of the cap covering a needle of the training drug delivery device, or (vi) an attachment of the needle to the training drug delivery device; and
transmit, using the wireless unit of the training drug delivery device to the user device, sensor indications corresponding to the user’s handling of the training drug delivery device.

26. The system of claim 25, wherein the user device is configured to indicate, in response to receiving the sensor indications, to the user whether the user’s handling of the training drug delivery device is correct.

27. The system of claim 25, wherein detecting the user’s handling of the training drug delivery device comprises detecting, using the at least one sensor, at least one of (i) the torque exerted on the dosage dial relative to the body of the training drug delivery device or (ii) the force exerted on the injection mechanism relative to the body of the training drug delivery device.

28. The system of claim 27, wherein detecting the user’s handling of the training drug delivery device comprises detecting, using the at least one sensor, at least one of (i) the attachment of the cap of the training drug delivery device covering at least a part of the body of the training drug delivery device, (ii) the attachment of the cap covering the needle of the training drug delivery device, or (iii) the attachment of the needle to the training drug delivery device.

29. The system of claim 16, wherein the training drug delivery device comprises a container configured to be filled with a liquid.

30. The system of claim 16, wherein the training drug delivery device is configured to receive a drug cartridge.

31. The system of claim 16, further comprising a training pad for simulating a user’s skin.

32. The system of claim 16, wherein the haptic response of the training drug delivery device is based on parameters of the haptic response received from the user device.

33. The system of claim 16, wherein the training drug delivery device further comprises a needle shield and a needle shield activation button.

34. A training drug delivery device comprising:
a body, a cap, a delivery activation button, a controller, a memory, a wireless unit for communicating with a user device, a drive for simulating a haptic response of a real drug delivery device, and at least one sensor for measuring an attachment of the cap and a depression of the delivery activation button;
the training drug delivery device being configured to connect, using the wireless unit, to the user device and transmit sensor indications to the user device.

35. A user device comprising:
a controller, a memory, and a wireless unit;
the user device being configured to:
connect, using the wireless unit, to a training drug delivery device;
receive sensor indications from the training drug delivery device;
transmit parameters of a haptic response to the training drug delivery device; and
provide, based at least partly on the received sensor indications, a user with feedback on using the training drug delivery device.
